(19) United States
(12) Patent Application Publication
(10) Pub. No.: US 2011/0135626 A1
(43) Pub. Date: Jun. 9, 2011

(54) Localized Chemical Lysis of Ocular Tissue

(75) Inventor: Steven W. Kovalcheck, Aliso Viejo, CA (US)

(73) Assignee: Alcon Research, LTD., Fort Worth, TX (US)

(21) Appl. No.: 12/633,473

(22) Filed: Dec. 8, 2009

(51) Int. Cl. A61K 38/48; A61P 27/02; A61F 9/007

(52) U.S. Cl. 424/94.63; 604/20; 604/21

(57) ABSTRACT

A high-intensity pulsed-electrical-field (HIPEF) apparatus chemically induces lysis of ocular tissue within a localized portion of an eye. Instead of broadly applying a lysis affecting solution (e.g., plasmin) to the eye, the apparatus delivers solution to only a portion of the eye. The apparatus then alters the effectiveness of at least some of the solution delivered by applying a HIPEF to that solution. In some embodiments, for example, the apparatus delivers a solution that does not substantially affect lysis of ocular tissue and then enhances the solution's effectiveness by applying a HIPEF. As the apparatus applies the HIPEF with high precision, the HIPEF only enhances the effectiveness of the solution within select and localized portions of the eye. The apparatus is especially advantageous for vitreoretinal surgery, whereby the apparatus may selectively induce lysis of vitreous tissue within a localized portion of the vitreous cavity, without significantly affecting adjacent retinal tissue.

### TECHNICAL FIELD

The present invention relates generally to the field of eye surgery and more particularly to methods and apparatus for localizing chemical lysis of ocular tissue during eye surgery using high-intensity pulsed electric fields.

### BACKGROUND

Techniques and apparatus for dissociation and removal of highly hydrated macroscopic volumes of proteinaceous tissue from the human eye have been previously disclosed. In particular, techniques for dissociation and removal of highly hydrated macroscopic volumes of proteinaceous tissue using rapid variable direction energy field flow fractionation have been disclosed by Steven W. Kovalcheck in U.S. patent application Ser. No. 11/608,877, filed 11 Dec. 2006 and titled “System For Dissociation and Removal of Proteinaceous Tissue” (hereinafter “the Kovalcheck application”), the entire contents of which are incorporated herein by reference.

The techniques disclosed in the Kovalcheck application were described in detail in terms of vitreoretinal surgery. However, those of ordinary skill in the art will readily understand that those techniques are applicable to medical procedures in other areas in the body of humans or animals. As explained in the Kovalcheck application, prior art procedures for vitreoretinal posterior surgery have relied for decades on mechanical or traction methods such as: 1) tissue removal with shear cutting probes (utilizing either a reciprocating or rotary cutter); 2) membrane transection using scissors, a blade, or vitreous cutters; 3) membrane peeling with forceps and picks; and 4) membrane separation with forceps and viscous fluids. While improvements in mechanisms, materials, quality, manufacturability, system support, and efficacy have progressed, many of the significant advancements in posterior intraocular surgical outcomes have been primarily attributable to the knowledge, fortitude, skill, and dexterity of the operating ophthalmic physicians.

Other prior art procedures for vitreoretinal posterior surgery have relied on chemical methods that manipulate vitreous tissue with an enzymatic agent (e.g., hyaluronidase, plasmin, or microplasmin). The enzymatic agent is injected into the posterior of the eye in an amount, concentration, and length of exposure sufficient to induce lysis of ocular tissue. The enzymatic agent cleaves to the vitreoretinal juncture, resulting in the potential for a more complete removal of vitreous tissue as compared to use of only the methods discussed above. In practice, however, inducing lysis of vitreous tissue in this way requires that the enzymatic agent be broadly applied to the entire posterior of the eye, including retinal tissue. Thus, although use of an enzymatic agent has the potential for a more complete removal of vitreous tissue, such use also risks substantial damage to the structure and function of the adjacent retinal tissue.

### SUMMARY

As described more fully below, embodiments of the present invention chemically induce lysis of ocular tissue within a localized portion of an eye during eye surgery. Instead of broadly applying a lysis affecting solution (e.g., plasmin) to the eye, the present invention delivers solution to only a portion of the eye. The present invention then alters the effectiveness of at least some of the solution delivered by applying a high-intensity pulsed electrical field (HIPEF) to that solution, using a HIPEF probe.

More particularly, a high-intensity pulsed electrical field (HIPEF) apparatus includes a HIPEF probe, an irrigation system, an aspiration system, and a high voltage pulse generator. The irrigation system delivers a lysis affecting solution to ocular tissue within a portion of the eye, via an irrigation channel in the HIPEF probe or a cannula independent from the HIPEF probe. The HIPEF probe then alters the effectiveness of at least some of the solution delivered, by applying a HIPEF generated by the high voltage pulse generator.

In some embodiments, for example, the irrigation system delivers a lysis affecting solution that does not substantially affect lysis of ocular tissue, but the HIPEF probe enhances the solution's effectiveness by applying the HIPEF. As the HIPEF probe applies the HIPEF with high precision, the HIPEF enhances the effectiveness of lysis affecting solution only within select and localized portions of the eye.

In other embodiments, the irrigation system delivers a lysis affecting solution that does substantially affect lysis of ocular tissue, and the HIPEF probe suppresses the solution's effectiveness by applying the HIPEF. Again, as the HIPEF probe applies the HIPEF with high precision, the HIPEF suppresses the effectiveness of lysis affecting solution only within select and localized portions of the eye.

Thus, regardless of whether the HIPEF probe enhances or suppresses the effectiveness of the lysis affecting solution, the irrigation system and the HIPEF probe operate to chemically induce lysis of ocular tissue only within select portions of the eye. The aspiration system then removes the lysed ocular tissue from that portion of the eye, e.g., by aspiration vacuum.

With the above described advantages, the present invention is particularly well suited in the context of vitreoretinal surgery or partial vitrectomy. For example, the present invention may selectively induce lysis of vitreous tissue within a localized portion of the vitreous cavity, without significantly affecting the adjacent retinal tissue.

Of course, those skilled in the art will appreciate that the present invention is not limited to the above features, advantages, contexts or examples, and will recognize additional features and advantages upon reading the following detailed description and upon viewing the accompanying drawings.

### BRIEF DESCRIPTION OF THE DRAWINGS

FIG. 1 is a perspective view of an exemplary high-intensity pulsed electric field (HIPEF) probe used for intraocular posterior surgery.

FIG. 2 is an enlarged perspective view of the tip of the probe of FIG. 1.

FIG. 3 is a schematic diagram of a high-intensity pulsed electric field (HIPEF) apparatus according to some embodiments of the invention.

FIGS. 4A-4C illustrate various embodiments for chemically inducing lysis of ocular tissue within a localized portion of an eye by altering the effectiveness of a lysis affecting solution applied thereto.

FIG. 5 is a logic flow diagram illustrating one embodiment of a method for chemically inducing lysis of ocular tissue within a localized portion of an eye.

### DETAILED DESCRIPTION

The present disclosure describes an apparatus and method for chemically inducing lysis of ocular tissue within a localized portion of an eye during eye surgery. In the context of vitreoretinal surgery, for example, the apparatus and method selectively induce lysis of vitreous tissue within a localized portion of the vitreous cavity, without significantly affecting the adjacent retinal tissue. By inducing lysis of vitreous tissue only within a localized portion of the eye, the apparatus and method can be used to safely improve removal of vitreous tissue.

In one embodiment, for instance, the apparatus and method safely improves the approach for removal of vitreous tissue described in the Kovalcheck application (U.S. patent application Ser. No. 11/608,877). The Kovalcheck application describes using a high-intensity pulsed-electrical-field (HIPEF) rather than classical mechanical means historically used to engage, decompose, and remove vitreous tissue. The application of such a rapidly changing electrical field causes a local temporary dissociation of the adhesive and structural relations in components of vitreous tissue, thereby enabling vitreous tissue to be detached from the retinal membrane and removed from the vitreous cavity.

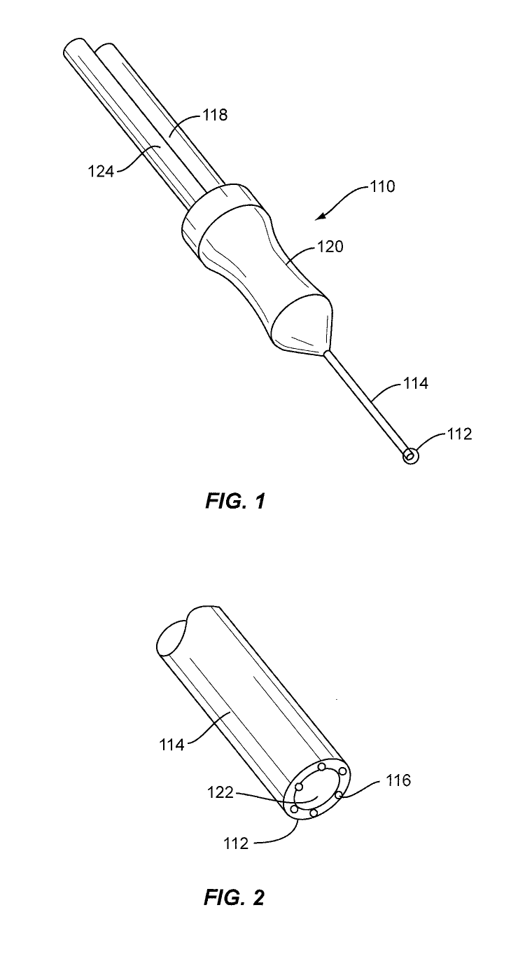

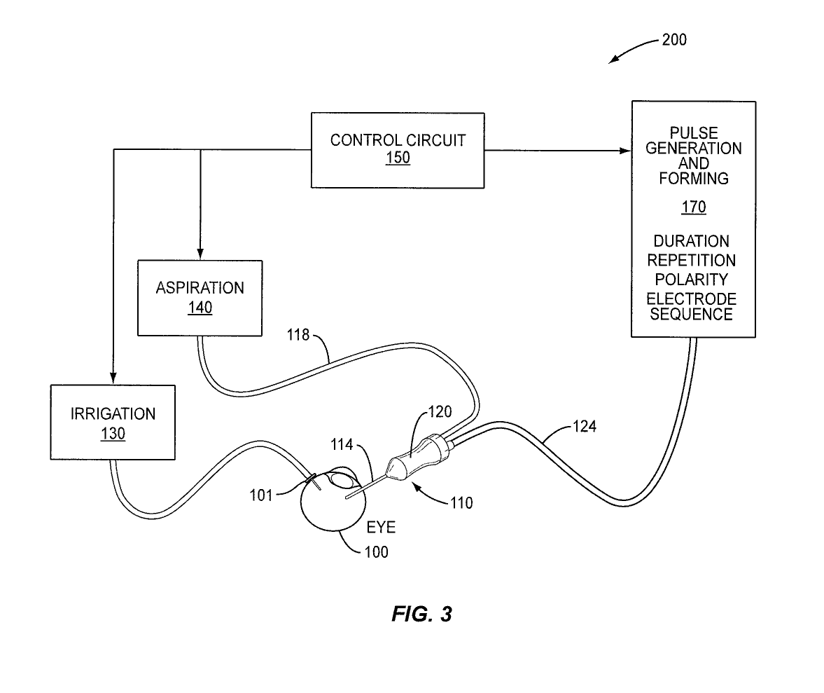

More particularly, the HIPEF is applied to vitreous tissue using a HIPEF probe 110 shown in FIG. 1. The HIPEF probe 110 comprises a hollow probe shaft 114 extending from a handle 120 to a probe shaft tip 112, as well as an aspiration line 118 and a transmission line 124. FIG. 2 illustrates details of the probe shaft 114 and probe shaft tip 112. As shown in FIG. 2, a plurality of electrodes 116 are exposed at the tip 112 and surround an aspiration lumen 122. The plurality of electrodes 116 are connected to the transmission line 124 for applying the HIPEF and disassociating vitreous tissue. The aspiration lumen 122 is connected to the aspiration line 118 for providing an aspiration pathway for disassociated vitreous tissue.

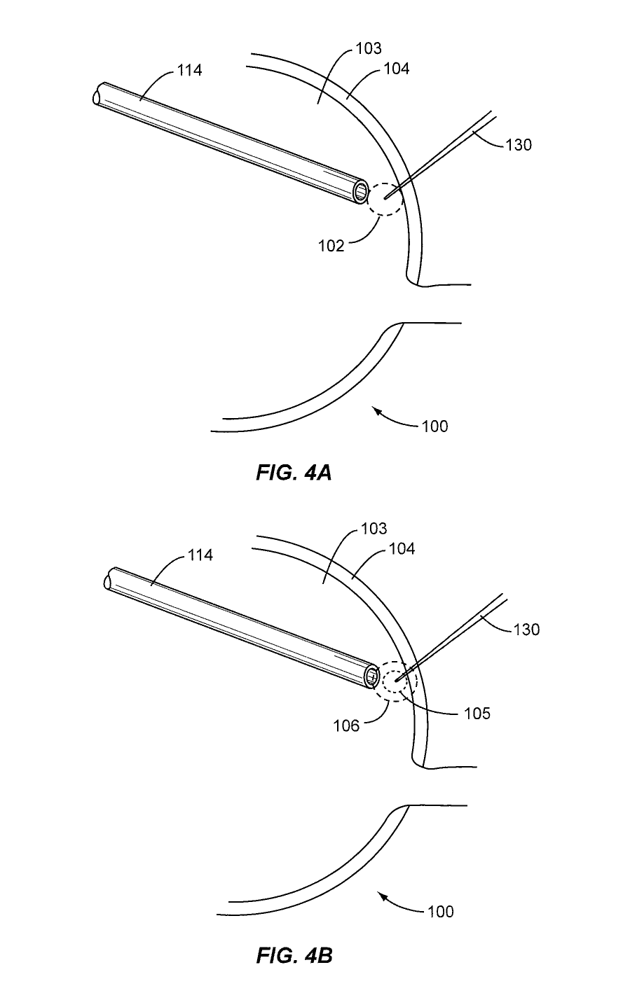

FIG. 3 illustrates additional operational details for applying a HIPEF to vitreous tissue with a HIPEF apparatus 200, which includes the HIPEF probe 110. Using handle 120, the tip 112 of the probe 110 may be inserted by a surgeon into the posterior region of an eye 100 via a pars plana approach 101. Using standard visualization processes, vitreous tissue is engaged by the tip 112 at the distal end of the hollow probe 114. An irrigation system 130 and an aspiration system 140 of the apparatus 200 are activated, by control circuit 150. The vitreous tissue is drawn into the orifice of the aspiration lumen 122 by the aspiration system 140, and then mixed with irrigation fluid delivered by the irrigation system 130 to control the electrical impedance of the vitreous tissue. Meanwhile, a high voltage pulse generator 170 of the apparatus 200 (which includes a pulse-forming network and switching circuit, in some embodiments) generates ultra-short high-intensity pulsed electric energy and sends that energy to the tip 112 via the transmission line 124 and electrodes 116. As the vitreous tissue traverses the high-intensity ultra-short-pulsed directionally changing electric field (HIPEF) concentrated at the tip 112, the adhesive mechanisms of the entrained volume of vitreous tissue are dissociated. The dissociated vitreous tissue is then removed from the vitreous cavity and drawn through the aspiration line 118 by the aspiration system 140 e.g., to a collection module.

To improve this approach as described in the Kovalcheck application and to provide for a more complete removal of vitreous tissue, the HIPEF apparatus 200 as described herein delivers irrigation fluid that consists wholly or partly of a lysis affecting solution. In one embodiment, for example, the lysis affecting solution is an enzymatic agent that chemically induces lysis of ocular tissue, such as vitreous tissue and even retinal tissue (e.g., hyaluronidase, plasmin, or microplasmin). However, to selectively induce lysis of vitreous tissue, without substantially affecting retinal tissue, the HIPEF apparatus 200 only delivers the lysis affecting solution to ocular tissue within a portion of the eye 100 and then alters the effect of at least some of the lysis affecting solution by applying a HIPEF to that solution.

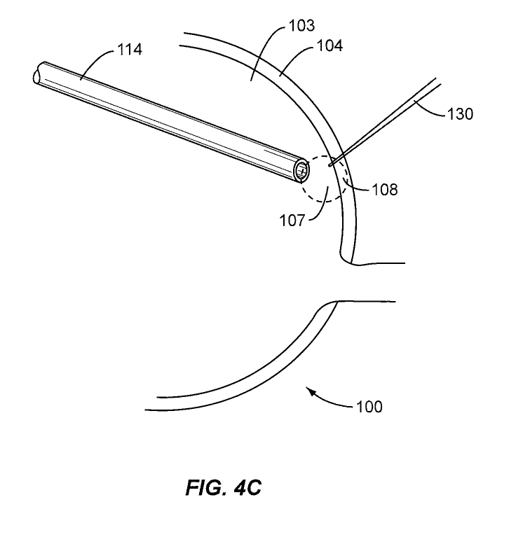

In the embodiment illustrated in FIG. 4A, for example, the irrigation system 130 is configured to deliver a lysis affecting solution to ocular tissue within a portion 102 of the eye 100. That is, instead of being broadly applied to the entire posterior region of the eye 100, including retinal tissue 104, the lysis affecting solution is substantially localized to ocular tissue within the portion 102 (i.e., vitreous tissue 103 within the portion 102). Delivered in this way and in this concentration and/or amount, the lysis affecting solution may not substantially affect lysis of the vitreous tissue 103. The HIPEF probe 110 in this embodiment, though, is configured to enhance the effectiveness of the lysis affecting solution by applying a HIPEF to the solution. So enhanced, the lysis affecting solution induces lysis of the vitreous tissue 103 and thereby improves the ability of the HIPEF apparatus 200 to dissociate and remove the vitreous tissue 103. Moreover, since the effectiveness of the lysis affecting solution is localized by the HIPEF apparatus 200 to the portion 102, this improvement safely dissociates vitreous tissue 103 i.e., without substantially affecting retinal tissue 104. Once dissociated, the lysed vitreous tissue 103 within the portion 102 may be removed as described above, e.g., via aspiration by the aspiration system 140.

Note that the HIPEF probe 110 primarily enables localized lysis of ocular tissue because of the highly precise and localized HIPEF, not necessarily the highly localized delivery of the lysis affecting solution. That is, the HIPEF apparatus 200 supports significantly more localized delivery of lysis affecting solution than the prior art (because it can alter the effectiveness of that solution), and thereby minimizes the risks to retinal tissue 104 inherent in broad application. Yet perfectly localized delivery of the solution to only vitreous tissue 103 may be substantially unavoidable e.g., due to diffusion of the solution to the retinal tissue 104. Nonetheless, the HIPEF probe 110 is configured to apply the HIPEF with high precision, so as to only alter the effectiveness of lysis affecting solution within select and localized portions of the eye 100 that contain vitreous tissue 103.

Take, for instance, the example in FIG. 4B. In FIG. 4B, the irrigation system 130 delivers the solution to ocular tissue within a portion 105 that is larger than the portion 102 shown in FIG. 4A, such that some of the solution is incidentally delivered to retinal tissue 104. Though delivered to the retinal tissue 104, the solution may not substantially affect lysis of the retinal tissue 104 without being enhanced. Accordingly, the HIPEF probe 110 applies a highly precise and localized HIPEF to enhance the effectiveness of only some of the lysis affecting solution delivered within the portion 105, namely the solution delivered to vitreous tissue 103 within the portion 106.

Although in the above embodiments the HIPEF apparatus 200 has been configured to enhance a lysis affecting solution's effectiveness by applying a HIPEF, the HIPEF apparatus 200 in other embodiments is configured to suppress the solution's effectiveness by applying that HIPEF. Consider the example in FIG. 4C. Much like the example in FIG. 4B, the irrigation system 130 in FIG. 4C delivers the solution to a relatively larger portion 108 such that some of the solution is incidentally delivered to retinal tissue 104. In this example, however, the irrigation system 130 delivers a lysis affecting solution that does substantially affect lysis of ocular tissue, including both vitreous tissue 103 and retinal tissue 104. Accordingly, instead of enhancing the effectiveness of solution delivered to the vitreous tissue 103, the HIPEF probe 110 is configured to suppress the effectiveness of solution delivered to the retinal tissue 104. That is, the HIPEF 110 applies a highly precise and localized HIPEF to suppress the effectiveness of only some of the lysis affecting solution delivered within the portion 107, namely the solution delivered to retinal tissue within the portion 108.

Regardless of whether the HIPEF probe 110 is configured to enhance or suppress the effectiveness of a lysis affecting solution, the HIPEF probe 110 generates the pulse shape, the pulse repetition rate, the pulse train length, and other parameters of the HIPEF based on the chemical properties of that solution. The parameters of a HIPEF for altering the effectiveness of a specific lysis affecting solution may be, for instance, pre-configured in the HIPEF apparatus 200 for that solution. The parameters of a HIPEF for altering the effectiveness of one or more different lysis affecting solutions may also be pre-configured in the HIPEF apparatus 200, whereby a surgeon may select between different pre-configurations based on the lysis affecting solution being delivered.

Moreover, FIGS. 1-4 above have illustrated the irrigation system 130 as being configured to deliver the lysis affecting solution by way of cannula independent from the HIPEF probe 110. However, those of ordinary skill in the art will understand that the irrigation system 130 may additionally or alternatively be configured to deliver the solution through one or more channels within the probe 110. Indeed, in such an embodiment, the HIPEF probe 110 may be configured to surround vitreous tissue within a portion of the eye 100, whereby the aspiration system 140 draws the tissue into a distal orifice of the probe via aspiration vacuum. The irrigation system 130 may then deliver the lysis affecting solution to the distal orifice of the probe 110 via an irrigation channel within the probe 110.

Furthermore, although the improved approach taught herein has been described above in the context of selectively inducing lysis of vitreous tissue, without affecting adjacent retinal tissue, those of ordinary skill in the art will understand the applicability of the disclosed invention for selectively inducing lysis of certain vitreous tissue without affecting other vitreous tissue. That is, the approach taught herein may also improve approaches for performing a partial vitrectomy. Generally, therefore, the particular ocular tissue to which the disclosed invention is directed does not limit the invention.

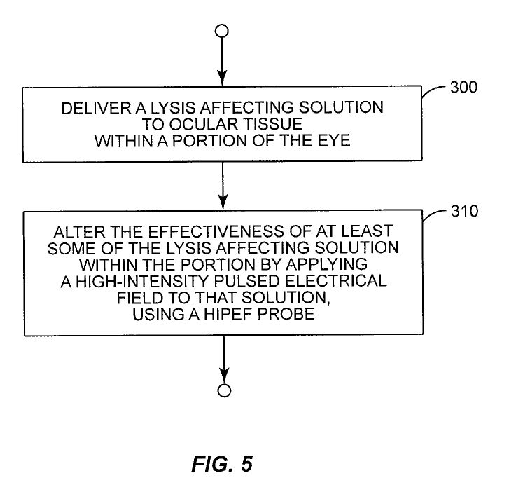

Accordingly, those of ordinary skill in the art will readily appreciate that the HIPEF apparatus 200 generally performs the method illustrated in FIG. 5 for chemically inducing lysis of ocular tissue within a localized portion of an eye 100. As shown in FIG. 5, the irrigation system 130 of the apparatus 200 delivers a lysis affecting solution to ocular tissue within a portion of the eye (Block 100). The HIPEF probe 110 then alters the effectiveness of at least some of the lysis affecting solution within the portion by applying a HIPEF to that solution.

Of course, this embodiment and all of the other embodiments described above for chemically inducing lysis of ocular tissue within a localized portion of an eye were given for purposes of illustration and example. Those skilled in the art will appreciate, therefore, that the present invention may be carried out in other ways than those specifically set forth herein without departing from essential characteristics of the invention. The present embodiments are thus to be considered in all respects as illustrative and not restrictive, and all changes coming within the meaning and equivalency range of the appended claims are intended to be embraced therein.

What is claimed is:

1. A method for chemically inducing lysis of ocular tissue within a localized portion of an eye during eye surgery, the method comprising:
delivering a lysis affecting solution to ocular tissue within a portion of an eye; and
altering the effectiveness of at least some of the lysis affecting solution within said portion by applying a high-intensity pulsed electrical field to said at least some solution, using a high-intensity pulsed-electrical-field (HIPEF) probe.

2. The method of claim 1, wherein delivering a lysis affecting solution comprises delivering an enzymatic agent that chemically induces lysis of ocular tissue.

3. The method of claim 1, wherein applying a high-intensity pulsed electric field to said at least some solution, using a high-intensity pulsed-electrical-field (HIPEF) probe, comprises generating the high-intensity pulsed electric field based on the chemical properties of the lysis affecting solution.

4. The method of claim 1, wherein altering the effectiveness of the lysis affecting solution by applying a high-intensity pulsed electrical field to solution within said portion of the eye comprises enhancing the effectiveness of at least some of the lysis affecting solution within said portion by applying a high-intensity pulsed electrical field to said at least some solution.

5. The method of claim 1, wherein altering the effectiveness of the lysis affecting solution by applying a high-intensity pulsed electrical field to solution within said portion of the eye comprises suppressing the effectiveness at least some of the lysis affecting solution within said portion by applying a high-intensity pulsed electrical field to said at least some solution.

6. The method of claim 1, wherein delivering a lysis affecting solution to ocular tissue within a portion of an eye comprises:
surrounding the ocular tissue with the HIPEF probe;
drawing the ocular tissue into a distal orifice of the HIPEF probe via aspiration vacuum; and
delivering the lysis affecting solution to the distal orifice of the probe via an irrigation channel in the HIPEF probe.

7. The method of claim 1, wherein delivering a lysis affecting solution to ocular tissue within a portion of an eye comprises delivering the lysis affecting solution to the ocular tissue via a cannula independent from the HIPEF probe.

8. The method of claim 1, further comprising removing lysed ocular tissue from said portion of the eye by aspiration.

9. The method of claim 1, wherein delivering a lysis affecting solution comprises delivering plasmin.

10. A high-intensity pulsed-electrical-field (HIPEF) apparatus for chemically inducing lysis of ocular tissue within a localized portion of an eye during eye surgery, the HIPEF apparatus comprising:
an irrigation system configured to deliver a lysis affecting solution to ocular tissue within a portion of an eye; and
a HIPEF probe configured to alter the effectiveness of at least some of the lysis affecting solution within said portion by applying a high-intensity pulsed electrical field to said at least some solution.

11. The HIPEF apparatus of claim 10, wherein the irrigation system is configured to deliver an enzymatic agent that chemically induces lysis of ocular tissue.

12. The HIPEF apparatus of claim 10, wherein the HIPEF probe is configured to generate the high-intensity pulsed electric field based on the chemical properties of the lysis affecting solution.

13. The HIPEF apparatus of claim 10, wherein the HIPEF probe is configured to enhance the effect of at least some of the lysis affecting solution within said portion by applying the high-intensity pulsed electrical field to said at least some solution.

14. The HIPEF apparatus of claim 10, wherein the HIPEF probe is configured to suppress the effect of at least some of the lysis affecting solution within said portion by applying the high-intensity pulsed electrical field to said at least some solution.

15. The HIPEF apparatus of claim 10, wherein the HIPEF probe is configured to surround the ocular tissue within said portion of the eye and to draw that ocular tissue into a distal orifice of the HIPEF probe via aspiration vacuum, and wherein the irrigation system comprises an irrigation channel in the HIPEF probe that is configured to deliver the lysis affecting solution to the distal orifice of the HIPEF probe.

16. The HIPEF apparatus of claim 10, wherein the irrigation system comprises a cannula independent from the HIPEF probe.

17. The HIPEF apparatus of claim 10, further comprising an aspiration system configured to remove lysed ocular tissue from said portion of the eye by aspiration.

18. The HIPEF apparatus of claim 10, wherein the irrigation system is configured to deliver plasmin.
